(19) United States
(12) Patent Application Publication
(10) Pub. No.: US 2014/0051934 A1
(43) Pub. Date: Feb. 20, 2014

(54) Stabilizing Port for Surgery for Facilitating Concurrent Introduction of Multiple Instruments

(71) Applicant: Covidien LP, Mansfield, MA (US)

(72) Inventors: Yong Ma, Cheshire, CT (US); Frank Viola, Sandy Hook, CT (US)

(21) Appl. No.: 13/940,288

(22) Filed: Jul. 12, 2013

Related U.S. Application Data
(60) Provisional application No. 61/683,739, filed on Aug. 16, 2012.

(51) Int. Cl. A61B 17/02

(52) U.S. Cl. CPC A61B 17/0218
USPC 600/208

(57) ABSTRACT

An access port assembly is provided including an access port having a proximal end and a distal end and a stabilizing anchor mechanically connected to the access port for stabilizing the access port within a tissue incision. An outer circumferential rim of the access port is configured and dimensioned to define a plurality of openings to facilitate concurrent introduction of a plurality of instruments.

### CROSS REFERENCE TO RELATED APPLICATION

The present application claims the benefit of and priority to U.S. Provisional Application Ser. No. 61/683,739, filed on Aug. 16, 2012, the entire contents of which are incorporated herein by reference.

### BACKGROUND

1. Technical Field

The present disclosure relates to an apparatus and method for accessing a body cavity. More particularly, the present disclosure relates to a stabilizing access port for facilitating concurrent introduction of multiple instruments.

2. Background of Related Art

Access assemblies configured for reception through an incision into an abdominal cavity are known, as are methods of inserting the access assemblies therethrough. Traditional access assemblies include a rigid cannula that is received through the tissue of the body wall into the body cavity. Endoscopic, laparoscopic and other suitable instruments may then be directed through a housing on the proximal end of the cannula to access the body cavity in a sealing manner in a variety of procedures.

Moreover, compressible assemblies configured for accessing a body cavity and permitting reception of electrosurgical instruments therethrough in a sealing manner are also known. Such compressible assemblies are composed of silicone, thermoplastic elastomers (TPE), rubber, foam, gel and other compressible materials and are configured to be compressed to facilitate insertion into an incision. Typically, such assemblies are deformed by a surgeon using his/her fingers or with the assistance of a grasping device, i.e., forceps. Compression of the assembly reduces the profile of the assembly, thereby facilitating reception of the assembly into the incision. Upon release of the compressive force, the compressed assembly returns to an uncompressed configuration. In the uncompressed configuration, the access assembly seals the opening into the body cavity. The assembly may have one or more access ports for receiving the instruments therethrough.

Therefore, it would be beneficial to have an access assembly configured to be inserted through tissue, such that surgical instruments may be easily inserted therethrough.

### SUMMARY

Accordingly, an access port assembly is provided. The access port assembly includes an access port having a proximal end and a distal end, and a stabilizing anchor mechanically connected to the access port for stabilizing the access port within a tissue incision. An outer circumferential rim of the access port is configured and dimensioned to define a plurality of openings to facilitate concurrent introduction of a plurality of instruments.

In one exemplary embodiment, the plurality of openings are equally spaced apart from each other. Alternatively, at least a portion of the plurality of openings abut each other. Additionally, the plurality of openings may be grouped into one or more abutting segments.

In yet another exemplary embodiment, the plurality of openings are of equal size. Alternatively, the plurality of openings have different sizes relative to each other.

In yet another exemplary embodiment, the access port includes two concentric circumferential rims positioned adjacent each other on the outer circumferential rim of the access port.

Moreover, the plurality of instruments may be wirelessly controlled. At least one of the plurality of instruments is a robotic arm.

In yet another exemplary embodiment, the distal portion of the outer circumferential rim includes inwardly angled and outwardly angled portions to maintain the surgical instruments in oblique positions when exiting the plurality of openings.

In another exemplary embodiment, a surgical instrument supporting device is provided. The surgical instrument supporting device includes a fixedly secured joint member and a supporting structure having a proximal end and a distal end, the supporting structure defining a plurality of passageways extending the length of the supporting structure. The plurality of passageways is located on an outer circumferential rim of the supporting structure, the plurality of passageways configured to receive surgical instruments therethrough.

The joint member may be fixedly secured to a stationary object.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective view of an access port assembly having a plurality of openings to facilitate concurrent introduction of a plurality of surgical instruments, in accordance with the embodiments of the present disclosure;

FIG. 2 is a perspective view of an access port including a plurality of openings, where at least a portion of the openings abut each other, in accordance with the embodiments of the present disclosure;

FIG. 3 is a perspective view of an access port including a plurality of openings, where the plurality of openings abut each other in separate and distinct groups, in accordance with the embodiments of the present disclosure;

FIG. 4 is a perspective view of an access port including a plurality of openings, where the plurality of openings are different sizes with respect to each other, in accordance with the embodiments of the present disclosure;

FIG. 5 is a perspective view of an access port including a plurality of openings, where the plurality of openings are inwardly angled and outwardly angled at a distal end of the access port to maintain the surgical instruments in oblique positions when exiting the plurality of openings, in accordance with the embodiments of the present disclosure; and

FIG. 6 is a perspective view of the access port, where the access port includes two concentric circumferential rims positioned adjacent each other on the outer circumferential rim of the access port, in accordance with the embodiments of the present disclosure.

### DETAILED DESCRIPTION

The access ports of the present disclosure, either alone or in combination with a cannula assembly, provide a substantially fluid-tight seal between a body cavity of a patient and the outside atmosphere. The access ports, or seal assemblies, of the present disclosure are configured to receive surgical instruments of varying diameter. Various surgical procedures contemplated include laparoscopic and arthroscopic surgical procedures.

The access ports of the present disclosure contemplate the introduction of various types of instrumentation adapted for insertion through a trocar and/or cannula assembly while maintaining a substantially fluid-tight interface about the instrument to help preserve the atmospheric integrity of a surgical procedure from gas and/or fluid leakage. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments” or “instrumentation.”

Embodiments of the presently disclosed apparatus will now be described in detail with reference to the drawings, in which like reference numerals designate identical or corresponding elements in each of the several views. As used herein, the term “distal” refers to that portion of the tool, or component thereof which is further from the user while the term “proximal” refers to that portion of the tool or component thereof which is closer to the user. While the use of the access assembly is often described herein as engaging an incision, it should be recognized that this is merely exemplary and is not intended to limit the use of the assembly in any way, but rather it should be recognized that the present disclosure is intended to be useable in all instances in situations in which the access assembly engages an incision, a naturally occurring orifice, or any other suitable opening.

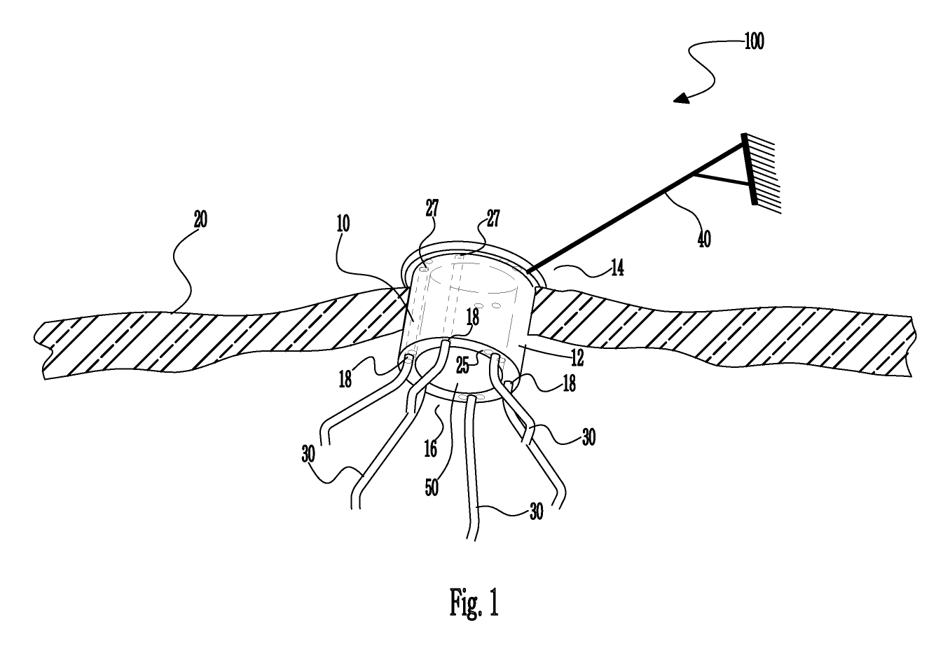

Referring to FIG. 1, there is disclosed an access port assembly 100 for use in single incision surgery. Access port assembly 100 is flexible or compressible to allow it to be inserted through a single incision or opening in the body of a patient such that after insertion it expands and seals within the incision. Additionally, the flexible nature of access port assembly 100 allows surgical instruments inserted therethrough to be manipulated relative to their respective axes and thus allows a relatively high degree of movement of the surgical instruments to orient them appropriate to the tissue being operated upon.

In particular, FIG. 1 depicts a perspective view of the access port assembly 100 having a plurality of openings 18 to facilitate concurrent introduction of a plurality of surgical instruments 30, in accordance with the embodiments of the present disclosure is presented.

The access port assembly 100 includes an access port 10 defining a tubular member 12. The tubular member 12 includes a proximal end 14 and a distal end 16. The proximal end 14 of the tubular member is fixedly secured within the tissue 20. The access port 10 is stabilized within the tissue 20 via a stabilizing anchor 40. The stabilizing anchor 40 may be fixedly secured to a stationary object. The access port 10 may include a central opening 50. The opening 50 may extend the length of the tubular member 12 of the access port 10. It is contemplated that additional surgical instruments may be inserted through opening 50. It is also contemplated that a surgeon's hand may be inserted through opening 50. It is also within the realm of the exemplary embodiments to close off the opening 50 during a number of surgical procedures. It is also within the scope of the present disclosure to position another surgical access port in opening 50. A suitable access port is fully described in U.S. patent application Ser. No. 12/244,024 filed Oct. 2, 2008, the entire contents of which are hereby incorporated by reference herein.

The distal end 16 includes a circumferential rim 25 configured and dimensioned to define a plurality of openings 18 to facilitate concurrent introduction of a plurality of instruments 30. The openings 18 are configured to extend through the outer wall of the tubular member 12 of the access port 10. Each of the plurality of instruments 30 may be inserted through the proximal end 14 of the access port 10 (collectively or independently) and may extend through the entire length of the outer wall or outer perimeter of the access port 10 to exit at openings 18. The plurality of channels 27 in the access port 10 are lumens or passages adapted to receive elongate surgical instruments 30. Thus, a surgeon may be able to simultaneously manipulate the plurality of surgical instruments 30 during a single surgical procedure, without having to constantly insert/remove instruments from the surgical field. Additionally, it is noted that each of the plurality of surgical instruments 30 may be independently operable.

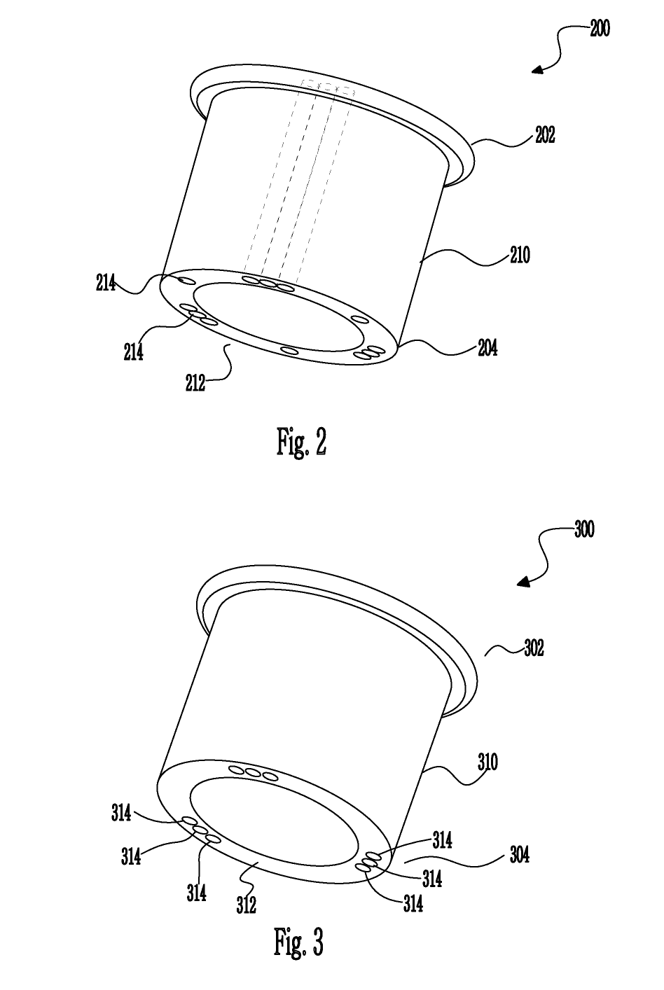

Additionally, in the configuration of FIG. 1, the plurality of surgical instruments 30 may be inserted through the proximal end 14 and exit at the plurality of openings 18 at the distal end 16 of the access port 10. In this exemplary embodiment, the openings 18 are equally spaced apart from each other. However, the positioning of the plurality of openings 18 may take on various configurations, as discussed below with reference to FIGS. 2-5.

The plurality of instruments 30 may be controlled by wired or wireless methodologies. Additionally, at least one of the pluralities of instruments 30 may be a robotic arm. Of course, all of the plurality of instruments 30 may be robotic arms.

The term “tubular” as used herein to describe access port 10 is intended to encompass various cross-sectional shapes, including round, oval, rectangular, and the like. Further, access port 10 may not have a continuous, solid wall, but instead may have longitudinal or circumferential slots or holes, or may have a cage or mesh structure with sufficient rigidity to maintain the shape and patency of the plurality of openings 18 when positioned in the tissue 20.

Access port 10 may be made of any of a variety of biocompatible materials. However, because these components may penetrate into a body cavity and come into contact with living tissue, they are usually sterile. Therefore, access port 10 is preferably constructed of a material, which may be sterilized between uses, or which is disposable after each use. Sterilizable materials suitable for access port 10 include stainless steel, anodized aluminum, and other biocompatible metals. Materials, which have the necessary performance characteristics, yet which are of sufficiently low cost to be disposable, include plastics such as polycarbonate, acetonitrile butyl styrene (ABS), or polyvinyl chloride (PVC).

In the exemplary embodiments of the present disclosure, maintaining the position of access port 10 is attained by a rigid or semi-rigid anchor member 40, which may be fixed to the operating table or other supporting structure or any other type of stationary object. Several different types of fixation configurations may be provided for adjustably attaching access port 10 to anchor member 40. In one embodiment, access port 10 may be attached to anchor member 40 by means of a collar (not shown) which clamps about the access port 10 near its proximal end, preferably about the upper rim. The collar may be pivotally attached to anchor member 40 by a ball-and-socket joint (not shown), which may be locked in a particular position by, for example, one or more set screws (not shown). In this way, access port 10 may be positioned in the body cavity and manipulated to a desired angular orientation so that the surgical site is visible through the plurality of passageways 18.

Additionally, the access port 10 of the present disclosure is capable of accommodating objects of varying diameters, e.g., including instruments from about 4.5 millimeter (mm) to about 15 millimeter (mm), during a minimally invasive surgical procedure. Moreover, the access port 10 enables the introduction and manipulation of various types of instrumentation adapted for insertion through a trocar and/or cannula assembly while maintaining a fluid tight interface about the instrumentation to prevent gas and/or fluid leakage from the established pneumoperitoneum so as to preserve the atmospheric integrity of a surgical procedure.

Referring to FIG. 2, a perspective view of an access port 200 including a plurality of openings 214, where at least a portion of the openings 214 abut each other, in accordance with the embodiments of the present disclosure is presented.

The access port 200 includes a tubular member 210 having a proximal end 202 and a distal end 204. The distal end 204 includes a circumferential rim 212 having the plurality of openings 214. As shown in FIG. 2, a portion of the openings 214, in select areas of the distal end 204, abut each other, while other openings 214 are separate and distinct from the abutting openings.

Referring to FIG. 3, a perspective view of an access port 300 including a plurality of openings 314, where the plurality of openings 314 abut each other in separate and distinct groups, in accordance with the embodiments of the present disclosure is presented.

The access port 300 includes a tubular member 310 having a proximal end 302 and a distal end 304. The distal end 304 includes a circumferential rim 312 having the plurality of openings 314. As shown in FIG. 3, the openings 314 abut each other, in certain select portions of the circumferential rim 312.

Referring to FIG. 4, a perspective view of an access port including a plurality of openings, where the plurality of openings are different sizes with respect to each other, in accordance with the embodiments of the present disclosure is presented.

The access port 400 includes a tubular member 410 having a proximal end 402 and a distal end 404. The distal end 404 includes a circumferential rim 412 having the plurality of openings 414, 416, 418. As shown in FIG. 4, the openings 414, 416, 418 have different sizes for accommodating instruments of varying diameters. For example, opening 414 is bigger than openings 416 and 418. Obviously, one skilled in the art may contemplate a plurality of different opening with different sizes positioned around the circumferential rim 412 of the access port 400.

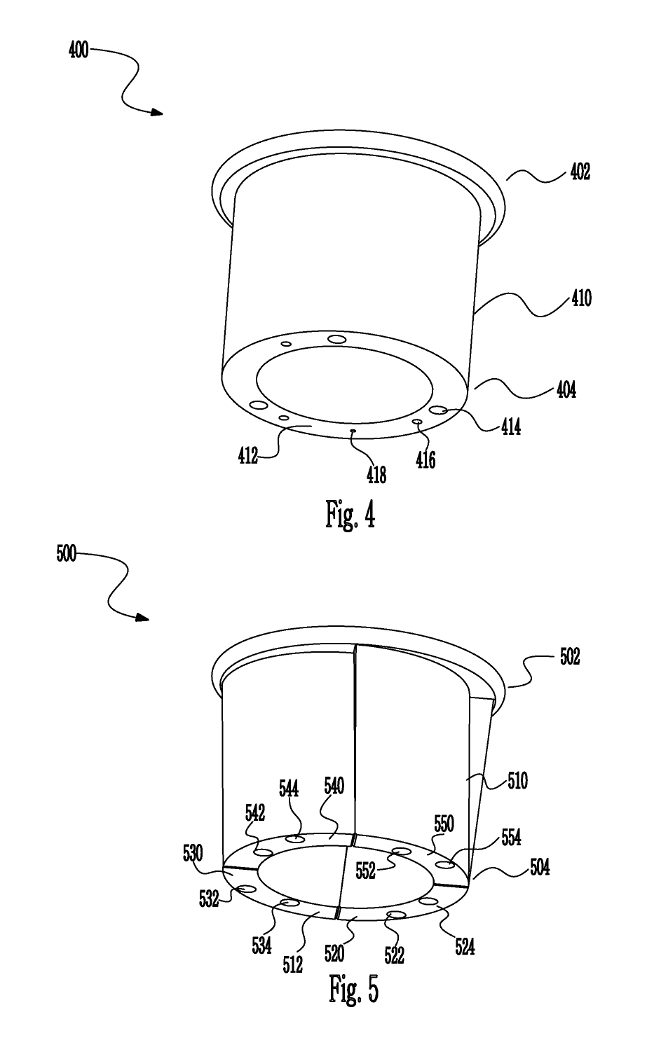

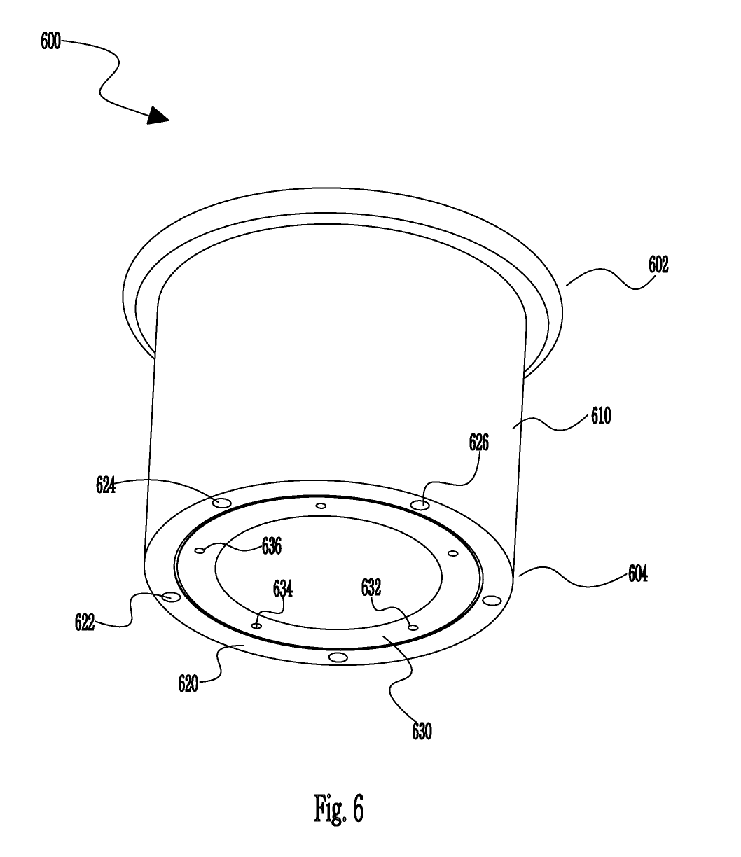

Referring to FIG. 5, a perspective view of an access port including a plurality of openings, where the plurality of openings are inwardly angled and outwardly angled at a distal end of the access port to maintain the surgical instruments in oblique positions when exiting the plurality of openings, in accordance with the embodiments of the present disclosure is presented.

The access port 500 includes a tubular member 510 having a proximal end 502 and a distal end 504. The distal end 504 includes a circumferential rim 512 having the plurality of openings that are grouped into one or more segments. As shown in FIG. 5, segments 520, 530 are outwardly angled, whereas segments 540, 550 are inwardly angled. Segment 520 includes openings 522 and 524, and segment 530 includes openings 532, 534. Additionally, segment 540, includes openings 542, 544, and segment 550 includes openings 552, 554. Thus, the distal end 504 of the outer circumferential rim 512 includes inwardly angled and outwardly angled portions to maintain the surgical instruments in oblique positions when exiting the plurality of openings.

Referring to FIG. 6, a perspective view of the access port 600, where the access port 600 includes two concentric circumferential rims 620, 630 positioned adjacent each other on the outer surface of the access port 600, in accordance with the embodiments of the present disclosure is presented.

The access port 600 includes a tubular member 610 having a proximal end 602 and a distal end 604. The distal end 604 includes two circumferential rims 620, 630 each having a plurality of openings disposed thereon. As shown in FIG. 6, the first circumferential rim 620 includes openings 622, 624, 626, whereas the second circumferential rim 630 includes openings 632, 634, 636. Thus, the access port 600 includes two concentric circumferential rims 620, 630 positioned adjacent each other on the outer surface of the access port 600, such that each circumferential rim 620, 630 includes a plurality of openings (e.g., 622, 624, 626, 632, 634, 636), equally spaced apart from each other. Of course, one skilled in the art may contemplate a plurality of abutting openings stretching around each rim 620, 630, where the openings may be of different sizes.

The advantages of the exemplary embodiments of the present disclosure as described herein may include a less extensive invasive procedure with shorter recovery time. The present disclosure may reduce the need for subsequent procedures of this nature to be performed. In addition, surgical practitioners may realize an improved rate of operational success through use of instrument devices, which provide means for real-time instrument manipulation, enhanced visual acuity, larger instrument size and selection options and reduced trauma to the patient.

The exemplary embodiments of the present disclosure provide the practitioner with a more versatile and functional laparoscopic surgical system and provide improved procedural techniques to enhance the surgeon's success rate. These improvements include at least decreasing the time required for a procedure and reducing the number of invasive penetrations, thus lowering patient trauma and risk.

The exemplary embodiments offer advantages where multiple conduits and/or larger conduits may be desirable, including concurrent real-time viewing, using multiple or larger instruments and improving instrument manipulation while concurrently diagnosing and observing the procedure continuously in real-time using video equipment. The multiple parallel channels or lumens allow instruments to be selectively inserted concurrently with each other, thus permitting real-time procedural visualization. It will be apparent to those skilled in the art that the configuration and relative positions of the conduits or lumens or passageways with respect to each other and the general shape of the access ports and related components is variable and may be tailored to procedural needs.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

1. An access port assembly, comprising:
an access port having a proximal end and a distal end; and
a stabilizing anchor mechanically connected to the access port for stabilizing the access port within a tissue incision;
wherein an outer circumferential rim of the access port is configured and dimensioned to define a plurality of openings to facilitate concurrent introduction of a plurality of instruments.

2. The access port assembly according to claim 1, wherein at least some of the plurality of openings are equally spaced apart from each other.

3. The access port assembly according to claim 1, wherein at least a portion of at least some of the plurality of openings abut each other.

4. The access port assembly according to claim 1, wherein at least some of the plurality of openings are grouped into one or more abutting segments.

5. The access port assembly according to claim 1, wherein at least some of the plurality of openings are of equal size.

6. The access port assembly according to claim 1, wherein the plurality of openings have different sizes relative to each other.

7. The access port assembly according to claim 1, wherein the access port includes two concentric circumferential rims positioned adjacent each other on the outer circumferential rim of the access port.

8. The access port assembly according to claim 1, further comprising at least one instrument, wherein the instrument is wirelessly controlled.

9. The access port assembly according to claim 1, further comprising at least one instrument, wherein the instrument is configured to interface with a robotic arm.

10. The access port assembly according to claim 1, wherein a distal portion of the outer circumferential rim includes inwardly angled and outwardly angled portions configured to maintain a surgical instruments in an oblique positions when exiting the plurality of openings.

11. A surgical instrument supporting device, comprising:
a fixedly secured joint member; and
a supporting structure having a proximal end and a distal end, the supporting structure defining a plurality of passageways extending the length of the supporting structure;
wherein the plurality of passageways is located on an outer circumferential rim of the supporting structure, the plurality of passageways configured to receive surgical instruments therethrough.

12. The surgical instrument supporting device according to claim 11, wherein the joint member is fixedly secured to a stationary object.

13. The surgical instrument supporting device according to claim 11, wherein at least some of the plurality of passageways are equally spaced apart from each other.

14. The surgical instrument supporting device according to claim 11, wherein at least a portion of at least some of the plurality of passageways abut each other.

15. The surgical instrument supporting device according to claim 11, wherein at least some of the plurality of passageways are grouped into one or more abutting segments.

16. The surgical instrument supporting device according to claim 11, wherein the supporting structure includes two concentric circumferential rims positioned adjacent each other on the outer circumferential rim of the supporting structure.

17. The surgical instrument supporting device according to claim 11, further comprising at least one surgical instrument, wherein the surgical instrument is wirelessly controlled.

18. The surgical instrument supporting device according to claim 11, further comprising at least one instrument, wherein the instrument is configured to interface with a robotic arm.

19. The surgical instrument supporting device according to claim 11, wherein a distal portion of the outer circumferential rim includes inwardly angled and outwardly angled portions configured to maintain a surgical instruments in oblique positions when exiting the plurality of passageways.
